(19) United States
(12) Patent Application Publication
(10) Pub. No.: US 2010/0285141 A1
(43) Pub. Date: Nov. 11, 2010

(54) COMPOSITIONS AND METHODS FOR ENHANCING THE ACTIVITY OF PODOPHYLLOTOXIN

(75) Inventor: Eran Gabbai, Kfar-MaAs (IL)

Correspondence Address: FOLEY AND LARDNER LLP;SUITE 500, 3000 K STREET NW, WASHINGTON, DC, 20007, US

(73) Assignee: DO-COOP TECHNOLOGIES LTD.

(21) Appl. No.: 12/735,314

(22) PCT Filed: Dec. 28, 2008
(86) PCT No.: PCT/IL08/01682

§ 371 Date: Jul. 1, 2010

Related U.S. Application Data
(60) Provisional application No. 61/006,268, filed on Jan. 3, 2008.

(51) Int. Cl. A61K 31/7028; A61K 9/14; A61K 31/366; C07H 15/24; C07D 493/04; A61P 35/00

(52) U.S. Cl. 424/490; 514/27; 514/463; 536/18.1; 549/298; 977/700; 977/915

(57) ABSTRACT

A method of increasing an activity of a podophyllotoxin or a derivative thereof is disclosed. The method comprises contacting the podophyllotoxin or the derivative with a liquid composition having a liquid and nanostructures, each of the nanostructures comprising a core material of the nanometric size surrounded by an envelope of ordered fluid molecules, the core material and the envelope of ordered fluid molecules being in a steady physical state, thereby increasing the activity of the podophyllotoxin or the derivative. Pharmaceutical compositions comprising same and uses thereof are also disclosed.

### FIELD AND BACKGROUND OF THE INVENTION

The present invention relates to a method for enhancing the activity of a podophyllotoxin or a derivative thereof and compositions comprising same.

Podophyllotoxin (podofilox) and its derivatives, etoposide and teniposide, are all cytostatic (antimitotic) glucosides.

Podofilox is an extract of the mandrake root Podophyllum peltatum which generally acts as a cell poison to cells undergoing mitosis (division). It is used in creams for the treatment for genital warts.

Etoposide is a semisynthetic derivative of podophyllotoxin. This drug is an important chemotherapeutic agent that is used to treat a wide spectrum of human cancers. It has been in clinical use for more than two decades and remains one of the most highly prescribed anticancer drugs in the world. The primary cytotoxic target for etoposide is topoisomerase II. This ubiquitous enzyme regulates DNA under- and overwinding, and removes knots and tangles from the genome by generating transient double-stranded breaks in the double helix. Etoposide kills cells by stabilizing a covalent enzyme-cleaved DNA complex (known as the cleavage complex) that is a transient intermediate in the catalytic cycle of topoisomerase II. The accumulation of cleavage complexes in treated cells leads to the generation of permanent DNA strand breaks, which trigger recombination/repair pathways, mutagenesis, and chromosomal translocations. If these breaks overwhelm the cell, they can initiate death pathways. Thus, etoposide converts topoisomerase II from an essential enzyme to a potent cellular toxin that fragments the genome.

Etoposide (which is sold by Bristol-Myers Squibb as VePesid, aka VP-16) is administered intravenously or orally as liquid capsules. It is used mainly to treat testicular cancer which hasn't responded to other treatment and as first-line treatment for small-cell lung cancers. It is also used to treat chorionic carcinomas, Kaposi's sarcoma, lymphomas and malignant melanomas. Major side effects include hair loss, nausea, anorexia, diarrhea, and low leukocyte and platelet counts.

Teniposide is used less often than etoposide; mainly, it is used to treat lymphomas. It has similar side effects.

### SUMMARY OF THE INVENTION

According to an aspect of some embodiments of the present invention there is provided a method of increasing an activity of a podophyllotoxin or a derivative thereof, the method comprising contacting the podophyllotoxin or the derivative with a liquid composition having a liquid and nanostructures, each of said nanostructures comprising a core material of a nanometric size surrounded by an envelope of ordered fluid molecules, said core material and said envelope of ordered fluid molecules being in a steady physical state, thereby increasing the activity of the podophyllotoxin or the derivative.

According to an aspect of some embodiments of the present invention there is provided a pharmaceutical composition comprising, as an active agent a podophyllotoxin or a derivative thereof and a composition having a liquid and nanostructures, each of said nanostructures comprising a core material of a nanometric size surrounded by an envelope of ordered fluid molecules, said core material and said envelope of ordered fluid molecules being in a steady physical state.

According to an aspect of some embodiments of the present invention there is provided a use of a liquid composition for the manufacture of a medicament comprising a podophyllotoxin or a derivative thereof, said liquid composition having a liquid and nanostructures, each of said nanostructures comprising a core material of a nanometric size surrounded by an envelope of ordered fluid molecules, said core material and said envelope of ordered fluid molecules being in a steady physical state, thereby increasing the activity of the podophyllotoxin or the derivative.

According to an aspect of some embodiments of the present invention there is provided a method of treating a hyperproliferative disease or HIV, the method comprising administering to a subject in need thereof the pharmaceutical composition of the present invention, thereby treating the disease.

According to some embodiments of the invention, the derivative is Etoposide or Teniposide.

According to some embodiments of the invention, at least a portion of said fluid molecules are identical to molecule of said liquid.

According to some embodiments of the invention, the at least a portion of said fluid molecules are in a gaseous state.

According to some embodiments of the invention, a concentration of said nanostructures is lower than 10²⁰ nanostructures per liter.

According to some embodiments of the invention, the nanostructures are capable of forming clusters of said nanostructures.

According to some embodiments of the invention, the nanostructures are capable of maintaining long range interaction thereamongst.

According to some embodiments of the invention, the liquid composition comprises a buffering capacity greater than a buffering capacity of water.

According to some embodiments of the invention, the liquid composition comprises a stable or meta-stable gas phase.

According to some embodiments of the present invention the nanostructure composition is capable of releasing the gas in response to excitation energy applied thereto and collecting the gas when the excitation energy is terminated.

According to some embodiments of the present invention the nanostructure composition is prepared in non-atmospheric conditions.

According to some embodiments of the present invention the nanostructure composition is prepared in the presence of a gas jet.

According to some embodiments of the present invention the nanostructure composition is prepared in the presence of gas at a concentration which is substantially different from natural atmospheric concentration of the gas.

According to some embodiments of the present invention the nanostructure composition is prepared in the presence of gas at a temperature which is substantially below an ambient temperature.

According to some embodiments of the present invention the envelope of fluid molecules is distinguishable from the liquid.

According to some embodiments of the present invention the gas phase comprises a hydrophobic gas.

According to some embodiments of the present invention the gas phase is selected from the group consisting of carbon dioxide, oxygen, nitrogen, sulfur dioxide, hydrogen, fluorine, methane, hexane, hexafluoroethane and air.

According to some embodiments of the present invention the gas phase resides in or attached to the envelope.

According to some embodiments of the present invention the gas phase resides in or attached to the core.

According to some embodiments of the present invention the gas phase resides in liquid regions between the nanostructures.

According to some embodiments of the invention, the liquid composition is capable of altering polarization of light.

According to some embodiments of the invention, the hyperproliferative disease is a cancer.

According to some embodiments of the invention, the cancer is selected from the group consisting of a chorionic carcinoma, Kaposi's sarcoma, lymphomas malignant melanoma, small cell lung cancer and testicular cancer.

According to some embodiments of the invention, the hyperproliferative disease is a genital wart.

According to some embodiments of the present invention the liquid comprises water.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE DRAWINGS

Some embodiments of the invention are herein described, by way of example only, with reference to the accompanying drawings and images. With specific reference now to the drawings and images in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-B are bar graphs illustrating that Neowater™ is capable of enhancing the activity of etoposide. LnCap (FIG. 1A) and PC3 prostate cancer cells (FIG. 1B) were incubated with the indicated concentration of Etoposide in Neowater™ or deionized water (as indicated) for 24 hours, and the number of surviving cells was measured by active uptake of XTT dye (an indicator of cell viability). ** The differences between each treated group were statistically significant (p<0.01).

FIG. 2 is a graph illustrating the optical activity of Neowater™ relative to DDW spectrum. The red and blue curves are measurements of different Neowater™ batches, measured at different dates.

FIG. 3 is a schematic illustration of a gas enriched nanostructure composition, according to various exemplary embodiments of the present invention.

FIG. 4 is a schematic illustration of gas recycling process, according to various exemplary embodiments of the present invention.

### DESCRIPTION OF THE PREFERRED EMBODIMENTS

The present invention relates to a method for enhancing the activity of a podophyllotoxin or a derivative thereof and compositions comprising same.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Whilst studying characteristics of compositions comprising nanostructures (such as described in U.S. Pat. Appl. Nos. 60/545,955, 10/865,955, 61/196,692 and International Patent Application, Publication No. WO2005/079153 and International Patent Application No. PCT/IL2008/001142, the contents of which are hereby incorporated by reference), the present inventor unexpectedly found that such compositions were capable of enhancing activity of podophyllotoxins, such as etoposide.

In particular, the present inventor discovered that a gas enriched nanostructure composition (GENC) can enhance activity of podophyllotoxins, e.g., etoposide. A gas enriched nanostructure composition is a composition containing nanostructures which is enriched with gas such that the gas remains in the composition.

As used herein the term “nanostructure” refers to a structure on the sub-micrometer scale which includes one or more particles, each being on the nanometer or sub-nanometer scale and commonly abbreviated “nanoparticle”. The distance between different elements (e.g., nanoparticles, molecules) of the structure can be of order of several tens of picometers or less, in which case the nanostructure is referred to as a “continuous nanostructure”, or between several hundreds of picometers to several hundreds of nanometers, in which the nanostructure is referred to as a “discontinuous nanostructure”. Thus, the nanostructure of the present embodiments can comprise a nanoparticle, an arrangement of nanoparticles, or any arrangement of one or more nanoparticles and one or more molecules. A typical size of a nanostructure in the nanostructure composition of the present embodiments is less than 100 nm, more preferably less than 50 nm, more preferably less than 40 nm, e.g., about 30 nm or about 20 nm.

A composition suitable for the present embodiments can be a nanostructure composition in which there is a stable or meta-stable gas phase.

A stable gas phase as used herein refers to a gas phase which remains in the liquid for a prolong period of time (from several days to several years, e.g., several months) without being spontaneously released to the environment.

A meta-stable gas phase refers to a gas phase which is spontaneously released from the liquid to the environment after a relatively short period of time (from several minutes to several days, e.g., several hours).

Spontaneous release of gas refers to a gas release which occurs when the liquid is in thermal equilibrium with the environment without any application of energy.

Without being bound to a specific theory, the present inventor postulates that stable gas phase of the composition can reside near the nanostructures or in spaces formed between the nanostructures. Such stable gas phase is typically in the form of nanobubbles. Yet, it is not excluded that the gas phase is trapped in a crystalline core of the nanostructures.

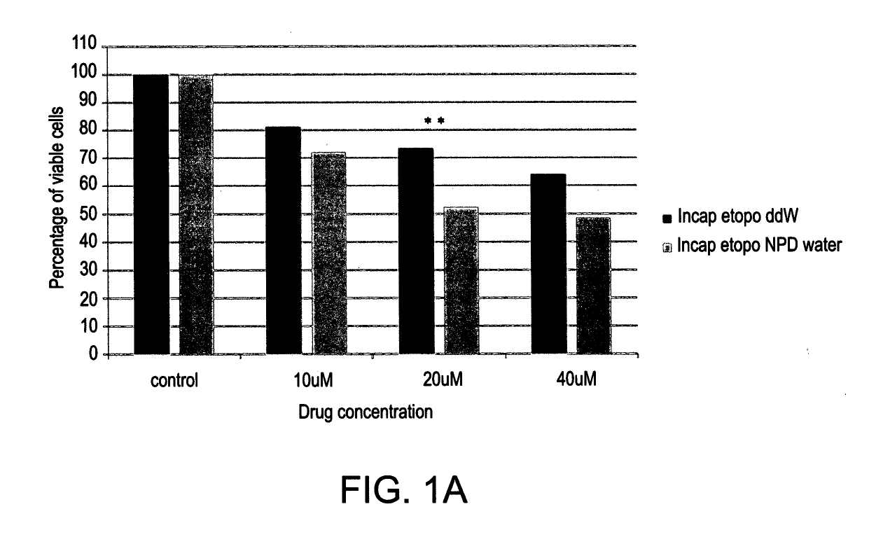

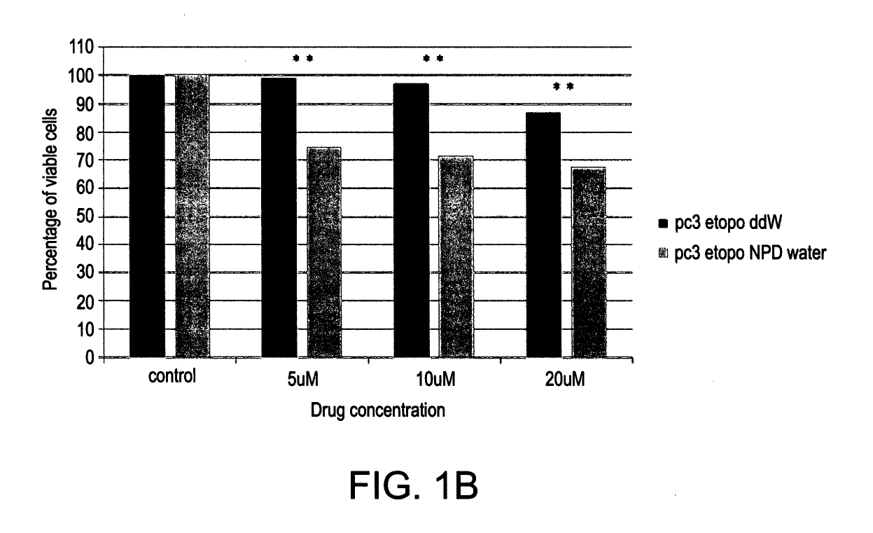

Whilst reducing the invention to practice the present inventors showed that etoposide formulated with compositions comprising nanostructures was more toxic for cancer cells as compared to etoposide formulated in standard deionized water (FIGS. 1A-B).

Thus, according to one aspect of the present invention, there is provided a method of increasing an activity of a podophyllotoxin or a derivative thereof, the method comprising contacting the podophyllotoxin or the derivative with a liquid composition having a liquid and nanostructures, each of the nanostructures comprising a core material of a nanometric size surrounded by an envelope of ordered fluid molecules, the core material and the envelope of ordered fluid molecules being in a steady physical state, thereby increasing the activity of the podophyllotoxin or the derivative.

As used herein, the term “podophyllotoxin” or “podofilox” refers to a cytostatic glucoside extracted from the mandrake root Podophyllum peltatum. Exemplary derivatives of podophyllotoxins include, but are not limited to etoposide and teniposide. The podophyllotoxin may be in any form (e.g. liquid, solid—such as powder).

The term “activity” as used herein, refers to a cellular activity, for example a cytostatic activity.

Contacting the podophyllotoxin with the liquid composition of the present invention may be effected by addition thereof or by replacement of a carrier composition in which the podophyllotoxin is dissolved. According to this embodiment, at least about 20% of the carrier composition comprises the liquid composition of the present invention, at least about 30%, at least about 40%, at least about 50%, at least about 60%, at least about 70%, at least about 80%, at least about 90% and at least about 100%. The addition of the liquid composition of the present invention to the podophyllotoxin may be effected using methods known in the art to increase solubility e.g. heating, stirring etc.

The liquid of the above-described composition is preferably an aquatic liquid e.g., water.

According to some embodiments of this aspect of the present invention the nanostructures of the liquid composition comprise a core material of a nanometer size enveloped by ordered fluid molecules, which are in a steady physical state with the core material and with each other. Such a liquid composition is described in U.S. Pat. Appl. Nos. 60/545,955, 10/865,955 and 61/196,692 and International Pat. Appl. Publication No. WO2005/079153 and International Pat. Appl. No. PCT/IL2008/001142 to the present inventor, the contents of which are incorporated herein by reference.

As used herein, the phrase “ordered fluid molecules” refers to an organized arrangement of fluid molecules which are interrelated, e.g., having correlations thereamongst. For example, instantaneous displacement of one fluid molecule can be correlated with instantaneous displacement of one or more other fluid molecules enveloping the core material.

The phrase “steady physical state” in the context of the relation between the core material and the envelope of ordered fluid molecules refers to a situation in which the core material is bound to the envelope by any potential having at least a local minimum. Representative examples, for such a potential include, without limitation, Van der Waals potential, Yukawa potential, Lenard-Jones potential and the like. Other forms of potentials are also contemplated.

Preferably, the ordered fluid molecules of the envelope are identical to the liquid molecules of the liquid composition. The fluid molecules of the envelope may comprise an additional fluid which is not identical to the liquid molecules of the liquid composition and as such the envelope may comprise a heterogeneous fluid composition.

Due to the formation of the envelope of ordered fluid molecules, the nanostructures of the present embodiment preferably have a specific gravity that is lower than or equal to the specific gravity of the liquid.

The fluid molecules may be either in a liquid state or in a gaseous state or a mixture of the two.

In various exemplary embodiments of the invention the liquid composition is a gas enriched nanostructure composition, e.g., the gas enriched nanostructure composition described in PCT/IL2008/001142.

In some embodiments of the invention the ordered fluid molecules enveloping the core are in gaseous state. In some embodiments of the present invention the envelope is distinguishable from the core. This can be validated, for example, using cryogenic-temperature transmission electron microscopy.

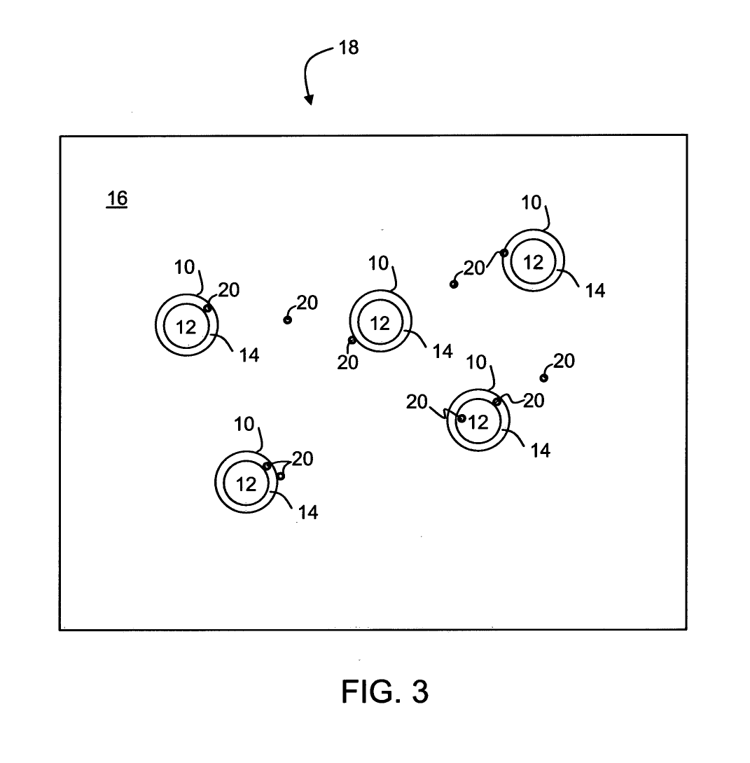

FIG. 3 is a schematic illustration of the liquid composition in embodiments in which the composition is a gas enriched nanostructure composition 18. Composition 18 comprises a nanostructure 10 and a liquid 16, such as, but not limited to, an aquatic, e.g., water. Also shown in FIG. 3 are a core material 12 and an envelope 14 of ordered fluid molecules, as further detailed hereinabove.

Composition 18 further comprises a gas phase 20, which resides in or attached to at least one of: envelope 14, core 12 and liquid 16. More specifically, the gas phase can (i) reside in the envelope, in which case the ordered fluid molecules also envelope the gas phase; (ii) reside in the boundary between the nanostructure and the liquid, in which case the gas phase is attached (e.g., bound) to the envelope, but is not surrounded thereby; (iii) reside in the solid structure (e.g., crystalline structure) of the core material, in which case the gas phase is trapped between atoms or molecules of the core material; and/or (iv) reside in liquid regions between the nanostructures. Any combination of these relations is also contemplated.

The gas phase can be in the form of gas molecules or is can be in a form of larger objects, such as, but not limited to, nanobubbles, which contain two or more gas molecules.

Many types of gasses are suitable for the gas enriched composition of the present embodiments. Typically, the gas is hydrophobic. Representative examples of suitable gases include, without limitation, carbon dioxide (CO₂), oxygen (O), nitrogen (N), sulfur dioxide (SO₂), hydrogen (H), fluorine (F), methane (CH₄), hexane (C₆H₁₄), hexafluoroethane (C₂F₆) and air. In various exemplary embodiments of the invention the gas is carbon dioxide (CO₂). In case of a CO₂ gas phase, the enriched composition of the present embodiments preferably has a relatively high pH values, indicating that the gas phase is not dissolved in the liquid.

The present inventor has discovered that an efflux of the gas can be generated by exciting the enriched nanostructure composition of the present embodiments to an excited state. The present inventor has uncovered that when the composition experience a transition from an excited state to a stable state, the composition act as a sink material for the gas. The present inventor has therefore uncovered that an influx of the gas can be generated by allowing excited compositions to return to their stable state.

Thus, in various exemplary embodiments of the invention the enriched nanostructure composition is characterized by at least a stable state and an excited state. The excited state is characterized by efflux of the gas from the composition, and a transition from the excited state to the stable state is accompanied by an influx of the gas to the composition.

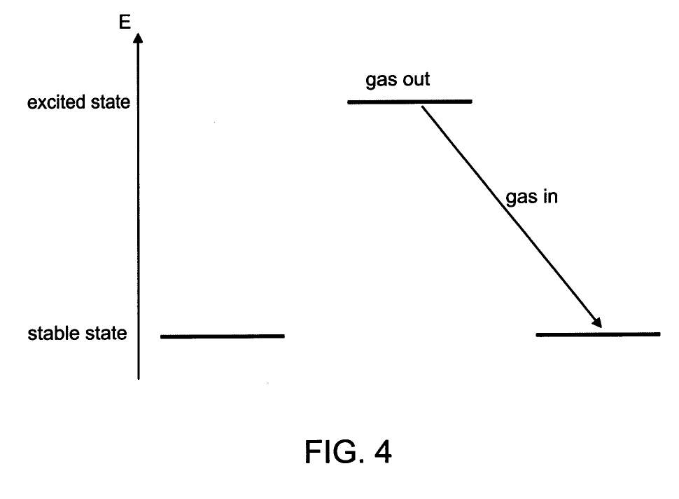

The process is illustrated in FIG. 4. As shown, the composition of the present embodiments generally has a stable state and an excited state. The term “generally” in the present context implies that the composition can have more than two states. For example, the composition may have one stable state and a plurality of excited states. The composition may also have a continuum of excited states. The term “excited state” in the present context refers to a state in which the energy of the composition is higher that the energy E when the composition is in its stable state.

The term “stable state” in the present context refers to a state in which the composition remains for a prolong period of time, substantially without experiencing spontaneous macroscopic transitions to another state. Typically, in the absence of severe conditions (such as delivery of vast amount of energy to the composition), the composition of the present embodiments can remain in its stable state for at least a day, more preferably at least a week, more preferably at least a month, more preferably at least a year, more preferably at least a few years.

Preferably, but not necessarily, the excited state (or states) of the composition are non-stable or meta-stable. When the excited state is non-stable, the transition from the excited state to the stable state is spontaneous, and there is no need to supply energy to the composition in order to achieve such transition. When the excited state is meta-stable, the transition from the excited state to the stable state can be achieved by supplying energy at a sufficient amount to perturb the composition to a non-stable state from which the composition spontaneously returns to the stable state.

Typically, the gas content of the composition is sufficiently high when the composition is in a stable state and is lower when the composition is in an excited state.

In various exemplary embodiments of the invention the composition is capable of generating a local concentration of gas, which is well above the normal ambient concentration.

In various exemplary embodiments of the invention the composition produces CO₂ at a local concentration of at least 2000 ppm by volume. Optionally and preferably, the composition produces CO₂ bursts at a local concentration of the order of 10,000 ppm by volume.

The core material of the composition of the present embodiments is not limited to a certain type or family of materials, and can be selected in accordance with the application for which the nanostructure is designed. Representative examples include, without limitation, ferroelectric material, a ferromagnetic material and a piezoelectric material. Also contemplated, is a core made of hydroxyapatite (HA). In some embodiments of the present invention the core material has a crystalline structure.

A ferroelectric material is a material that maintains, over some temperature range, a permanent electric polarization that can be reversed or reoriented by the application of an electric-field. A ferromagnetic material is a material that maintains permanent magnetization, which is reversible by applying a magnetic field. According to some embodiments of the present invention, when the core material is ferroelectric or ferromagnetic, the nanostructure retains its ferroelectric or ferromagnetic properties. Hence, the nanostructure has a particular feature in which macro scale physical properties are brought into a nanoscale environment.

According to some embodiments of the present invention the nanostructure is capable of clustering with at least one additional nanostructure. More specifically, when a certain concentration of the nanostructure is mixed in a liquid (e.g., water), attractive electrostatic forces between several nanostructures may cause adherence thereamongst so as to form a cluster of nanostructures. Preferably, even when the distance between the nanostructures prevents cluster formation, the nanostructure is capable of maintaining long range interaction (about 0.5-10 μm), with the other nanostructures.

As used herein the term “about” refers to ±10%.

A preferred concentration of the nanostructures is below 10²⁰ nanostructures per liter and more preferably below 10¹⁵ nanostructures per liter. Preferably a nanostructure in the liquid is capable of clustering with at least one additional nanostructure due to attractive electrostatic forces between them. Preferably, even when the distance between the nanostructures prevents cluster formation (about 0.5-10 μm), the nanostructures are capable of maintaining long-range interactions.

Production of the nanostructures according to this aspect of the present invention may be carried out using a “top-down” process. The process comprises the following method steps, in which a raw powder (e.g., a mineral, a ceramic powder, a glass powder, a metal powder, or a synthetic polymer) of micro-sized particles, say, above 1 μm or above 10 μm in diameter, which are broken in a controlled manner, to provide nanometer-sized particles. Typically, such a process is performed in a cold liquid (preferably, but not obligatory, water) into which high-temperature raw powder is inserted, under condition of electromagnetic radiofrequency (RF) radiation.

Examples of solid powders which are contemplated include, but are not limited to, BaTiO₃, WO₃ and Ba₂F₉O₁₂. Surprisingly, the present inventors have shown that hydroxyapetite (HA) may also be heated to produce the liquid composition of the present invention. Hydroxyapatite is specifically preferred as it is characterized by intoxocicty and is generally FDA approved for human therapy.

It will be appreciated that many hydroxyapatite powders are available from a variety of manufacturers such as Sigma Aldrich and Clarion Pharmaceuticals (e.g. Catalogue No. 1306-06-5).

In some embodiments of the invention an enriched nanostructure composition is manufactured in non-atmospheric conditions. In some embodiments of the present invention, the enriched nanostructure composition is manufactured in the presence of gas which is not naturally present in the atmosphere and/or in the presence of gas whose concentration is substantially higher (e.g., at least two times higher, or at least ten time higher, or at least a hundred times higher) or substantially lower (e.g., at least two times lower, or at least ten time lower, or at least a hundred times lower) than its natural concentration.

For example, the enriched nanostructure composition of the present embodiments can be manufactured in the presence of carbon dioxide at a concentration which is above the atmospheric concentration of carbon dioxide. The atmospheric concentration of carbon dioxide is typically less than 400 ppm by volume. Thus, in some embodiments of the present invention the enriched nanostructure composition is manufactured in the presence of carbon dioxide at a concentration of at least 400 ppm, or at least 600 ppm or or at least 800 ppm.

In some embodiments of the present invention the enriched nanostructure composition is manufactured in the presence of gas jet. In some embodiments of the present invention the enriched nanostructure composition is manufactured in the presence of gas, preferably gas jet, at a temperature which is substantially below the ambient temperature.

A solid powder (e.g., a mineral, a ceramic powder, a glass powder, a metal powder, a synthetic polymer, etc.) can be heated to a sufficiently high temperature (for example, about 700° C. or more, e.g., about 850° C.), and subsequently immersed in a cold liquid in the presence of a gas medium. In some embodiments of the present invention the liquid is water below its density anomaly temperature, e.g., 3° C. or 2° C. Substantially contemporaneously with the immersion, the cold liquid, powder and gas medium are irradiated by electromagnetic radiofrequency radiation, e.g., 500 MHz, 750 MHz or more.

The gas phase can be introduced to the composition in more than one way. In some embodiments of the present invention, the heated powder is passed through a gas medium, typically a flow of gas medium, prior to the immersion of the powder in the cold liquid. In some embodiments of the present invention, a gas medium is introduced into the cold liquid prior to or substantially contemporaneously with the immersion. The gas medium is introduced to the liquid in the form of bubbles.

The gas medium is preferably hydrophobic. Representative examples of suitable gas media include, without limitation, carbon dioxide (CO₂), oxygen (O), nitrogen (N), sulfur dioxide (SO₂), hydrogen (H), fluorine (F), methane (CH₄), hexane (C₆H₁₄), hexafluoroethane (C₂F₆) and air. In various exemplary embodiments of the invention the gas medium is carbon dioxide (CO₂).

It has been demonstrated by the present inventor that during the production process described above, some of the large agglomerates of the source powder disintegrate and some of the individual particles of the source powder alter their shape and become spherical nanostructures. It is postulated [Katsir et al., “The Effect of rf-Irradiation on Electrochemical Deposition and its Stabilization by Nanoparticle Doping”, Journal of The Electrochemical Society, 154(4) D249-D259, 2007] that during the production process, nanobubbles are generated by the radiofrequency treatment and cavitation is generated due to the injection of hot particles into water below the anomaly temperature. Since the water is kept below the anomaly temperature, the hot particles cause local heating that in turn leads to a local reduction of the specific volume of the heated location that in turn causes under pressure in other locations. It is postulated that during the process and a time interval of a few hours or less following the process, the water goes through a self-organization process that includes an exchange of gases with the external atmosphere and selective absorption of the surrounding electromagnetic radiation. It is further postulated that the self-organization process leads to the formation of the stable structured distribution composed of the nanobubbles and the nanostructures.

As demonstrated Examples 2 hereinunder, the liquid composition of the present embodiments is characterized by a non-vanishing circular dichroism signal. Circular dichroism is an optical phenomenon that results when a substance interacts with plane polarized light at a specific wavelength. Circular dichroism occurs when the interaction characteristics of one polarized-light component with the substance differ from the interaction characteristics of another polarized-light component with the substance. For example, an absorption band can be either negative or positive depending on the differential absorption of the right and left circularly polarized components for the substance.

It is recognized that non-vanishing circular dichroism signal of the liquid composition indicates that the liquid composition is an optically active medium. Thus, the liquid composition of the present embodiments can alter the polarization of light while interacting therewith. The present inventor postulates that the optical activity of the liquid composition of the present embodiments is a result of the long-range order which is manifested by the aforementioned formation of stable structured distribution of nanobubbles and nanostructures.

Since podophyllotoxins are used to treat hyperproliferative disorders and HIV, compositions comprising same together with nanostructures as described herein may be used to treat patients suffering from such diseases.

Thus, according to an aspect of the invention, there is provided a method of treating a hyperproliferative disorder and HIV, the method comprising administering to a subject in need thereof a therapeutically effective amount of a pharmaceutical composition comprising, as an active agent a podophyllotoxin or a derivative thereof and a composition having a liquid and nanostructures, each of the nanostructures comprising a core material of a nanometric size surrounded by an envelope of ordered fluid molecules, the core material and the envelope of ordered fluid molecules being in a steady physical state.

As used herein the term “subject in need thereof” refers to a mammal, preferably a human subject.

As used herein the term “treating” refers to preventing, curing, reversing, attenuating, alleviating, minimizing, suppressing or halting the deleterious effects of a hyperproliferative disease or HIV.

Types of hyperproliferative diseases amenable to treatment via the method of the present invention include benign tumors, warts (e.g. genital warts), polyps, precancers, and malignant tumors/cancer.

As used herein the term “cancer” refers to the presence of cells possessing characteristics typical of cancer-causing cells, for example, uncontrolled proliferation, loss of specialized functions, immortality, significant metastatic potential, significant increase in anti-apoptotic activity, rapid growth and proliferation rate, and certain characteristic morphology and cellular markers. In some circumstances, cancer cells will be in the form of a tumor; such cells may exist locally within an animal, or circulate in the blood stream as independent cells, for example, leukemic cells.

Specific examples of cancer which can be treated using the compositions of the present invention include, but are not limited to, adrenocortical carcinoma, hereditary; bladder cancer; breast cancer; breast cancer, ductal; breast cancer, invasive intraductal; breast cancer, sporadic; breast cancer, susceptibility to; breast cancer, type 4; breast cancer, type 4; breast cancer-1; breast cancer-3; breast-ovarian cancer; Burkitt's lymphoma; cervical carcinoma; colorectal adenoma; colorectal cancer; colorectal cancer, hereditary nonpolyposis, type 1; colorectal cancer, hereditary nonpolyposis, type 2; colorectal cancer, hereditary nonpolyposis, type 3; colorectal cancer, hereditary, nonpolyposis, type 6; colorectal cancer, hereditary nonpolyposis, type 7; dermatofibrosarcoma protuberans; endometrial carcinoma; esophageal cancer; gastric cancer, fibrosarcoma, glioblastoma multiforme; glomus tumors, multiple; hepatoblastoma; hepatocellular cancer; hepatocellular carcinoma; leukemia, acute lymphoblastic; leukemia, acute myeloid; leukemia, acute myeloid, with eosinophilia; leukemia, acute nonlymphocytic; leukemia, chronic myeloid; Li-Fraumeni syndrome; liposarcoma, lung cancer; lung cancer, small cell; lymphoma, non-Hodgkin's; lynch cancer family syndrome II; male germ cell tumor; mast cell leukemia; medullary thyroid; medulloblastoma; melanoma, meningioma; multiple endocrine neoplasia; myeloid malignancy, predisposition to; myxosarcoma, neuroblastoma; osteosarcoma; ovarian cancer; ovarian cancer, serous; ovarian carcinoma; ovarian sex cord tumors; pancreatic cancer; pancreatic endocrine tumors; paraganglioma, familial nonchromaffin; pilomatricoma; pituitary tumor, invasive; prostate adenocarcinoma; prostate cancer; renal cell carcinoma, papillary, familial and sporadic; retinoblastoma; rhabdoid predisposition syndrome, familial; rhabdoid tumors; rhabdomyosarcoma; small-cell, cancer of lung; soft tissue sarcoma, squamous cell carcinoma, head and neck; T-cell acute lymphoblastic leukemia; Turcot syndrome with glioblastoma; tylosis with esophageal cancer; uterine cervix carcinoma, Wilms' tumor, type 2; and Wilms' tumor, type 1, and the like.

According to one embodiment of this aspect of the present invention, the cancer is chorionic carcinoma, Kaposi's sarcoma, lymphomas malignant melanoma, small cell lung cancer and testicular cancer.

The compositions of the present invention are typically formulated as pharmaceutical compositions in order to treat diseases.

As used herein a “pharmaceutical composition” refers to a preparation of one or more of the active ingredients described herein (e.g. etoposide) with other chemical components such as physiologically suitable carriers and excipients. The purpose of the pharmaceutical composition is to facilitate administration of the active ingredients to the subject.

Hereinafter, the phrases “physiologically acceptable carrier” and “pharmaceutically acceptable carrier” which may be interchangeably used refer to a carrier or a diluent that does not cause significant irritation to the subject and does not abrogate the biological activity and properties of the administered active ingredients. An adjuvant is included under these phrases.

Herein, the term “excipient” refers to an inert substance added to the pharmaceutical composition to further facilitate administration of an active ingredient of the present invention. Examples, without limitation, of excipients include calcium carbonate, calcium phosphate, various sugars and types of starch, cellulose derivatives, gelatin, vegetable oils and polyethylene glycols. The pharmaceutical composition may advantageously take the form of a foam or a gel.

Techniques for formulation and administration of drugs may be found in “Remington's Pharmaceutical Sciences,” Mack Publishing Co., Easton, Pa., latest edition, which is incorporated herein by reference.

Suitable routes of administration include any of various suitable systemic and/or local routes of administration.

Suitable routes of administration may, for example, include the inhalation, oral, buccal, rectal, transmucosal, topical, transdermal, intradermal, transnasal, intestinal and/or parenteral routes; the intramuscular, subcutaneous and/or intramedullary injection routes; the intrathecal, direct intraventricular, intravenous, intraperitoneal, intranasal, and/or intraocular injection routes; and/or the route of direct injection into a tissue region of the subject.

The pharmaceutical composition may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with the present invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer.

For transmucosal administration, penetrants appropriate to the barrier to be permeated are used in the formulation. Such penetrants are generally known in the art.

For oral administration, the pharmaceutical composition can be formulated readily by combining the active ingredients with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active ingredient doses.

Pharmaceutical compositions which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration via the inhalation route, the active ingredients for use according to the present invention can be delivered in the form of an aerosol/spray presentation from a pressurized pack or a nebulizer with the use of a suitable propellant, e.g., a fluorochlorohydrocarbon such as dichlorodifluoromethane, trichlorofluoromethane, dichlorotetrafluoroethane; carbon dioxide; or a volatile hydrocarbon such as butane, propane, isobutane, or mixtures thereof. In the case of a pressurized aerosol, the dosage unit may be determined by providing a valve to deliver a metered amount. Capsules and cartridges of, e.g., gelatin for use in a dispenser may be formulated containing a powder mix of the active ingredients and a suitable powder base such as lactose or starch.

The pharmaceutical composition may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

A pharmaceutical composition for parenteral administration may include an aqueous solution of the active ingredients in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredients may be in powder form for constitution with a suitable vehicle, e.g., sterile, pyrogen-free water based solution, before use.

The pharmaceutical composition may also be formulated in rectal compositions such as suppositories or retention enemas, using, e.g., conventional suppository bases such as cocoa butter or other glycerides.

The pharmaceutical composition should contain the active ingredients in an amount effective to achieve disease treatment.

Determination of a therapeutically effective amount is well within the capability of those skilled in the art, especially in light of the detailed disclosure provided herein.

For any preparation used in the methods of the invention, the therapeutically effective amount or dose can be estimated initially from in vitro and cell culture assays. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p. 1).

Dosage amount and interval may be adjusted individually to provide plasma or brain levels of the active ingredients which are sufficient to achieve the desired therapeutic effect (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until cure is effected or diminution of the disease state is achieved.

The amount of the composition to be administered will be dependent on the subject being treated, the severity of the affliction, the manner of administration, the judgment of the prescribing physician, etc.

Compositions of the present invention may, if desired, be presented in a pack or dispenser device, such as an FDA approved kit, which may contain one or more unit dosage forms containing the active ingredients. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

The terms “comprises”, “comprising”, “includes”, “including”, “having” and their conjugates mean “including but not limited to”. This term encompasses the terms “consisting of” and “consisting essentially of”.

The phrase “consisting essentially of” means that the composition or method may include additional ingredients and/or steps, but only if the additional ingredients and/or steps do not materially alter the basic and novel characteristics of the claimed composition or method.

As used herein, the singular form “a”, “an” and “the” include plural references unless the context clearly dictates otherwise. For example, the term “a compound” or “at least one compound” may include a plurality of compounds, including mixtures thereof.

As used herein the term “about” refers to ±10%.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions, illustrate the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N.Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Example 1

### Enhancement of Etoposide Using Neowater™

Etoposide has extensive applications in the treatment of small cell lung cancer, advanced testicular cancer and Karposi's sarcoma.

The following experiment was performed in order to ascertain whether Neowater™ is capable of enhancing the activity of etoposide phosphate (hydrophilic analog of etoposide) and Etoposide-Teva® (pharmaceutical formulation of Etoposide). Two human prostate cancer cell lines were examined—LnCap and PC3.

Results

As shown in FIGS. 1A-B, etoposide causes a dose-dependent decrease in the proliferation of these cancer cells. PC3 and LnCap cell growth was significantly impaired by etoposide treatment. Etoposide formulated with Neowater was more toxic for both cancer cell lines as compared to etoposide formulated in standard deionized water.

### Example 2

### Optical Activity of Neowater™

Polarimetry measurements on the Neowater™ were devised to test signatures of induced long range order. The optical activity (in terms of circularly and elliptically polarized light) of the NPD solutions was measured using the Circular Dichroism (CD) method.

The Circular Dichroism (CD) experimental procedure: CD spectroscopy aims to detect absorption differences between left-handed and righthanded (L and R) polarized lights passed through aqueous solutions. Such differences can be generated from optically active (chiral) molecules immersed in water, distribution of molecules or nanoparticles or any other induced ordered structures in the water or solutions. The measurements reported here were performed using a Jasco K851 CD polarimeter at room temperature (298K). The spectrum was scanned between 190 nm and 280 nm using 1 nm and 10 seconds increments. In order to increase sensitivity and resolution a very long optical pathway was ensured by using 10 cm quartz cuvette (compared to 1 mm or smaller in regular mode of operation).

Results

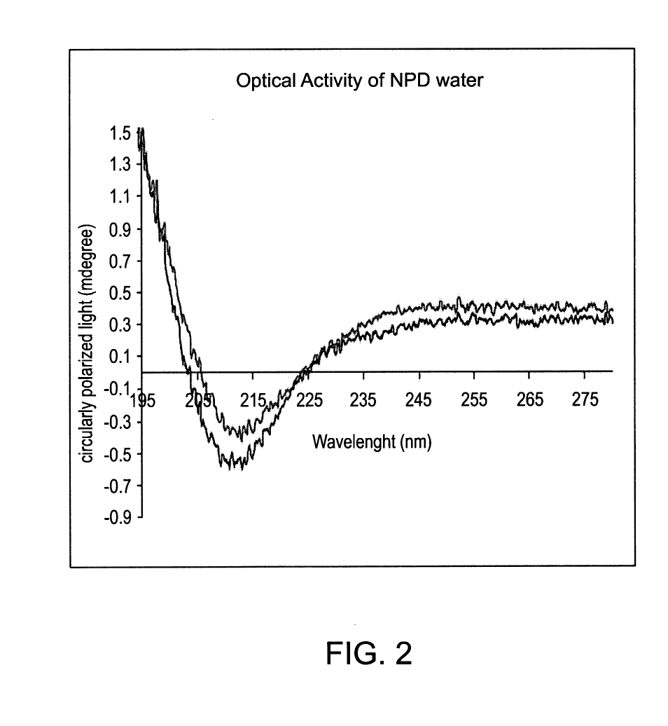

The results indicate that the Neowater™ shows circular dichroism. Two typical CD spectra performed in different batches of Neowater™, relative to the CD spectra of DDW (that was used as the baseline), are shown in FIG. 2. It is noted that the detected magnitude of the optical activity of about 0.5 millidegree is similar to the effect of 10⁵-10⁶ mole of ordinary peptide solution. Hence it is not a negligible level. CD measured differences in the absorption of left-handed polarized light versus right-handed polarized light arise due to structural asymmetry—The absence of regular structure results in vanishing CD intensity, while an ordered structure results in a spectrum which can contain positive and/or negative signals. Therefore, the present inventors propose that the existence of non vanishing signal in the CD spectra of the NPD solutions might be associated with the formation of long range orientational order in the Neowater™, formed by the network of nanoparticles and nanobubbles.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

1. A method of increasing an activity of a podophyllotoxin or a derivative thereof, the method comprising contacting the podophyllotoxin or the derivative with a liquid composition having a liquid and nanostructures, each of said nanostructures comprising a core material of a nanometric size surrounded by an envelope of ordered fluid molecules, said core material and said envelope of ordered fluid molecules being in a steady physical state, thereby increasing the activity of the podophyllotoxin or the derivative.

2. The method of claim 1, wherein the derivative is Etoposide or Teniposide.

3. The method of claim 1, wherein at least a portion of said fluid molecules are identical to molecule of said liquid.

4. The method of claim 1, wherein at least a portion of said fluid molecules are in a gaseous state.

5. The method of claim 1, wherein said liquid composition comprises a buffering capacity greater than a buffering capacity of water.

6. The method of claim 1, wherein said liquid composition is capable of altering polarization of light.

7. The method of claim 1, wherein said liquid composition comprises a stable or meta-stable gas phase.

8. (canceled)

9. The method of claim 1, wherein said liquid composition is prepared in the presence of a gas jet.

10. The method of claim 1, wherein said liquid composition is prepared in the presence of gas at a concentration which is substantially different from natural atmospheric concentration of said gas.

11. The method of claim 1, wherein said envelope of fluid molecules is distinguishable from said liquid.

12. The method of claim 1, wherein said core material is crystalline.

13-17. (canceled)

18. A pharmaceutical composition comprising, as an active agent a podophyllotoxin or a derivative thereof and a composition having a liquid and nanostructures, each of said nanostructures comprising a core material of a nanometric size surrounded by an envelope of ordered fluid molecules, said core material and said envelope of ordered fluid molecules being in a steady physical state.

19. The pharmaceutical composition of claim 18, wherein at least a portion of said fluid molecules are identical to molecule of said liquid.

20. The pharmaceutical composition of claim 18, wherein at least a portion of said fluid molecules are in a gaseous state.

21. The pharmaceutical composition of claim 18, wherein said liquid composition comprises a buffering capacity greater than a buffering capacity of water.

22. The pharmaceutical composition of claim 18, wherein said derivative is Etoposide or Teniposide.

23. The pharmaceutical composition of claim 18, wherein said liquid composition is capable of altering polarization of light.

24. The pharmaceutical composition of claim 18, wherein said liquid composition comprises a stable or meta-stable gas phase.

25. The pharmaceutical composition of claim 24, wherein said liquid composition is capable of releasing said gas in response to excitation energy applied thereto and collecting said gas when said excitation energy is terminated.

26. The pharmaceutical composition of claim 18, wherein said liquid composition is prepared in the presence of a gas jet.

27. The pharmaceutical composition of claim 18, wherein said liquid composition is prepared in the presence of gas at a concentration which is substantially different from natural atmospheric concentration of said gas.

28. The pharmaceutical composition of claim 18, wherein said envelope of fluid molecules is distinguishable from said liquid.

29. The pharmaceutical composition of claim 18, wherein said core material is crystalline.

30-35. (canceled)

36. A method of treating a hyperproliferative disease or HIV, the method comprising administering to a subject in need thereof a pharmaceutical composition comprising, as an active agent a podophyllotoxin or a derivative thereof, a liquid and nanostructures, wherein each of said nanostructures comprises a core material of a nanometric size surrounded by an envelope of ordered fluid molecules, said core material and said envelope of ordered fluid molecules being in a steady physical state, thereby treating the disease.

37. The method of claim 36, wherein the hyperproliferative disease is a cancer.

38-39. (canceled)
